(19) United States
(12) Patent Application Publication
(10) Pub. No.: US 2013/0073016 A1
(43) Pub. Date: Mar. 21, 2013

(54) HEATING DEVICE

(71) Applicant: Atsushi Matsuo, Ibaraki-shi (JP)

(72) Inventor: Atsushi Matsuo, Ibaraki-shi (JP)

(73) Assignee: KOBAYASHI PHARMACEUTICAL CO., LTD, Osaka-shi (JP)

(21) Appl. No.: 13/384,823

(22) PCT Filed: Dec. 4, 2009
(86) PCT No.: PCT/JP2009/070431

§ 371 Date: Jan. 19, 2012

(30) Foreign Application Priority Data
Sep. 28, 2009 (JP) 2009-223285

(51) Int. Cl. A61F 7/03; B23P 25/00

(52) U.S. Cl. 607/109; 607/111; 607/112; 29/527.2

(57) ABSTRACT

An object of the invention is to provide a heating device that is securely held in its worn position, and that can exhibit a sufficient heating effect.

A heating device comprising:
a heat-generating portion having an exothermic composition enclosed in one or more sections, the exothermic composition generating heat by contact with air; and
an elastic band portion;
(1) the band portion surrounding all or a portion of the heat-generating portion to keep the heat-generating portion in contact with the body;
(2) the band portion being connected to an end of the heat-generating portion;
(3) a face of the heat-generating portion that is brought into contact with the band portion having a moisture permeability of 340 to 610 g/m² per day, as measured according to Method A (Humidity Sensor Method) defined in JIS K7129; and
(4) the band portion having an air permeability of 3 s/300 cc to 61 s/300 cc, as measured according to JIS P 8117-1998: “Paper and board—Determination of air permeance and air resistance: Gurley method”.

### TECHNICAL FIELD

The present invention relates to heating devices, and, in particular, to therapeutic or rehabilitation devices for the wrist, ankle, knee, elbow, or neck.

### BACKGROUND ART

Heating devices with a heat-generating portion have been previously proposed as therapeutic or rehabilitation devices. Such heating devices generate heat when an exothermic composition inside the heat-generating portion comes into contact with air, thereby imparting a heating effect to an affected area.

For example, Patent Documents 1 and 2 disclose heating devices in the form of cylindrical supporters holding a chemical warmer. These supporters are worn on an affected area, such as the wrist or ankle, and impart a heating effect to the affected area by the heat generation of the chemical warmer.

However, when the supporters of Patent Documents 1 and are worn for many hours, they may be shifted from their original positions by, for example, the movement of the wearer or by contact with objects. In such cases, the area to be treated or rehabilitated may not be able to receive a sufficient heating effect.

Patent Document 3 discloses a cold protection device in the form of a cloth, wherein a disposable chemical warmer is held in a muffler. This cold protection device (muffler) is used around the neck, and thus does not easily shift from the position being warmed.

However, when the cold protection device (muffler) of Patent Document 3 is used around the neck, the outer side of the chemical warmer (i.e., the face not in contact with the body) is covered with the cloth; therefore, the exothermic composition inside the chemical warmer cannot be sufficiently contacted with air. Moreover, the muffler is usually wound in layers when it is used, making the contact of the exothermic composition with air even more difficult. For this reason, the muffler may not provide a desired heating effect because of insufficient heat generation of the chemical warmer inside the muffler.

Thus, there is a desire for the development of a heating device that is securely held in its worn position, and that can exhibit a sufficient heating effect.

### CITATION LIST

### Patent Literature

[PTL 1] Japanese Unexamined Patent Publication No. 2007-14792
[PTL 2] Japanese Unexamined Utility Model Publication No. 1989-62820
[PTL 3] Japanese Unexamined Patent Publication No. 2002-146612

### SUMMARY OF INVENTION

### Technical Problem

A principal object of the invention is to provide a heating device that is securely held in its worn position, and that can exhibit a sufficient heating effect.

### Solution to Problem

The present inventors conducted extensive research, and consequently found that the above-mentioned object can be achieved by providing a band portion for surrounding all or a portion of a heat-generating portion to keep the heat-generating portion in contact with the body, and by setting the moisture permeability of a face of the heat-generating portion that is brought into contact with the band portion within a specific range, and setting the air permeability of the band portion within a specific range.

The term “moisture permeability” as used herein means the value of the water vapor permeability. The water vapor permeability represents the amount of water vapor that passes through a test specimen of a unit area per day at a predetermined temperature and humidity.

The invention relates to heating devices as summarized below.

1. A heating device comprising an exothermic composition that generates heat by contact with air; a heat-generating portion that encloses the exothermic composition in one or more sections; and an elastic band portion;

(1) the band portion surrounding all or a portion of the heat-generating portion to keep the heat-generating portion in contact with the body;

(2) the band portion being connected to an end of the heat-generating portion;

(3) a face of the heat-generating portion that is brought into contact with the band portion having a moisture permeability of 340 to 610 g/m² per day, as measured according to Method A (Humidity Sensor Method) defined in JIS K7129; and

(4) the band portion having an air permeability of 3 s/300 cc to 61 s/300 cc, as measured according to JIS P 8117-1998: “Paper and board—Determination of air permeance and air resistance: Gurley method”.

2. The heating device according to Item 1, wherein the band portion is made of a nonwoven fabric.

3. The heating device according to Item 1 or 2, wherein the exothermic composition contains an iron powder, a water-retaining agent, a metal salt, and water.

4. The heating device according to any one of Items 1 to 3, wherein the exothermic composition contains 30 to 80 mass % of the iron powder, 2 to 30 mass % of the water-retaining agent, 0.5 to 10 mass % of the metal salt, and 1 to 40 mass % of the water; and the total amount of these components in the exothermic composition is 80 to 100 mass %.

5. The heating device according to any one of Items 1 to 4, wherein the face of the heat-generating portion that is brought into contact with the band portion has a moisture permeability of 365 to 475 m² per day.

6. The heating device according to any one of Items 1 to 5, wherein the band portion has an air permeability of 5 s/300 cc to 8 s/300 cc.

7. The heating device according to any one of Items 1 to 6, which is a therapeutic or rehabilitation device for a thumb side of a wrist joint, a wrist, an ankle, a knee, an elbow, or a neck.

8. A method for manufacturing a heating device comprising:

forming a heat-generating portion by laminating a first sheet that is non-breathable and a second sheet that is breathable so that an exothermic composition that generates heat by contact with air is enclosed in one or more sections; and

connecting an elastic band portion to an end of the heat-generating portion formed in the previous step so that the elastic band portion encloses all or a portion of the heat-generating portion, allowing the heat-generating portion to be kept in contact with the body;

a face of the heat-generating portion that is brought into contact with the band portion having a moisture permeability of 340 to 610 g/m² per day, as measured according to Method A (Humidity Sensor Method) defined in JIS K7129; and

the band portion having an air permeability of 3 s/300 cc to 61 s/300 cc, as measured according to JIS P 8117-1998: “Paper and board—Determination of air permeance and air resistance: Gurley method”.

### Advantageous Effects of Invention

The heating device of the invention allows the heat-generating portion to come into contact with the body, thereby imparting a heating effect to the affected area. Because the heat-generating portion is fixed with the elastic band portion, the heating device of the invention is not easily shifted from the affected area (the worn position) by the movement of the wearer, or by contact with objects. For this reason, the heating device can stably impart a heating effect to the affected area for many hours.

The heating device of the invention can advantageously impart moisturizing effects when the moisture permeability of the face of the heat-generating portion that is brought into contact with the band portion is within the above-specified numerical range, and the air permeability of the band portion is within the above-mentioned numerical range.

Known heating devices tend to result in an insufficient heating effect when the heat-generating portion is completely covered with another member. Furthermore, with known heating devices, covering a portion of the heat-generating portion with another member results in differences in the exothermic temperature and the rate of formation of water vapor between the uncovered portion and the portion covered with the other member. Consequently, the heating and moisturizing effects tend to be nonuniform depending on the part of the heat-generating portion. In contrast, the heating device of the invention is capable of providing uniform heating and moisturizing effects throughout the heat-generating portion, even though all or a portion of the heat-generating portion is covered with the band portion. The heating device of the invention has thus overcome the drawbacks of the known heating devices.

### BRIEF DESCRIPTION OF DRAWINGS

FIG. 1 is a diagram showing an embodiment of the use of the heating device of the invention.

FIG. 2 is a diagram showing an embodiment of the use of the heating device of the invention.

FIG. 3 is a schematic diagram of the heating device of the invention.

FIG. 4 is a diagram showing an example of the apparatus used for measuring the moisture permeability.

FIG. 5 is a diagram showing an example of the apparatus used for measuring the air permeability.

### DESCRIPTION OF EMBODIMENTS

The heating device of the invention includes an exothermic composition that generates heat by contact with air; a heat-generating portion that encloses the exothermic composition in one or more sections; and an elastic band portion; wherein:

(1) the band portion surrounds all or a portion of the heat-generating portion to keep the heat-generating portion in contact with the body;

(2) the band portion is connected to an end of the heat-generating portion;

(3) a face of the heat-generating portion that is brought into contact with the band portion has a moisture permeability of 340 to 610 g/m² per day, as measured according to Method A (Humidity Sensor Method) defined in JIS K7129; and

(4) the band portion has an air permeability of 3 s/300 cc to 61 s/300 cc, as measured according to JIS P 8117-1998: “Paper and board—Determination of air permeance and air resistance: Gurley method”.

The heating device of the invention imparts a heating effect to the affected area by bringing the heat-generating portion into contact with the body. The heat-generating portion is fixed with the elastic band portion. This prevents the heating device of the invention from being easily shifted from the affected area (the worn position) by the movement of the wearer, or by contact with objects. Therefore, the heating device of the invention can stably impart a heating effect to the affected area for many hours.

The term “heating effect” as used herein means warming the affected area by generating heat. The heating effect can, for example, improve the blood circulation of the affected area, and promote the restoration of the affected area while removing waste products from the tissue of the affected area. The heating effect allows the affected area to be warmed to about 38 to 42° C., and preferably about 39 to 41° C.

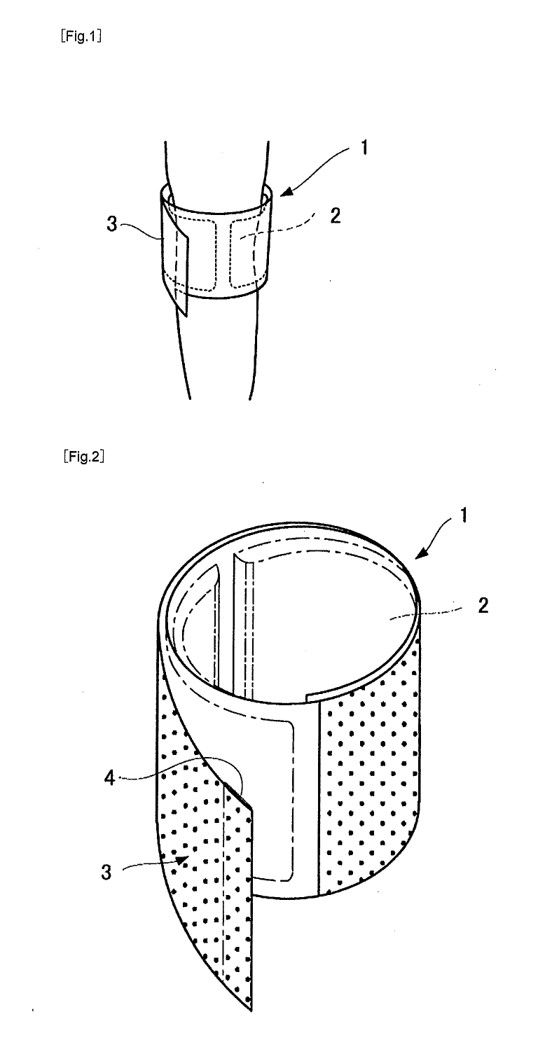

As shown in FIGS. 1 and 2, the heat-generating portion is fixed by winding the band portion around the body. Upon winding the band portion, the heat-generating portion is surrounded by one or more layers (preferably one layer) of the band portion.

Consequently, the band portion surrounds all or a portion of the heat-generating portion. That is, at least a portion of the heat-generating portion is covered with the band portion. The proportion of the region where the heat-generating portion is brought into contact with the band portion is preferably about 10 to 100%. If the proportion of the region in contact with the band portion is less than about 10%, the heat-generating portion often cannot be sufficiently fixed to the affected area. The region can be measured by, for example, determining the area of the band portion in contact with the heat-generating portion using a ruler.

The moisture permeability of the face of the heat-generating portion that is brought into contact with the band portion is about 340 to 610 g/m² per day, and preferably about 365 to 475 g/m² per day, as measured according to Method A (Humidity Sensor Method) defined in JIS K7129. If the moisture permeability is less than 340 g/m² per day, the affected area cannot be sufficiently warmed. If the moisture permeability exceeds 610 g/m² per day, the exothermic temperature of the exothermic composition will be too high, making the contact with the body difficult. Particularly, when the moisture permeability is about 365 to 475 g/m² per day, the affected area can be advantageously warmed to about 39 to 41° C.

The air permeability of the band portion is 3 s/300 cc to 61 s/300 cc, preferably about 3 to 8 s/300 cc, more preferably about 5 to 8 s/300 cc, and particularly preferably about 7 s/300 cc, as measured according to JIS P 8117-1998: “Paper and board—Determination of air permeance and air resistance: Gurley method”. When the air permeability is within these ranges, the heating effect can be effectively demonstrated.

When the moisture permeability of the heat-generating portion and the air permeability of the band portion are each within the above-defined ranges, a good heating effect can be achieved even when the heat-generating portion is completely covered with the band portion. Further, even when a portion of the heat-generating portion is covered with the band portion, a good heating effect can be achieved without producing nonuniformity in the heating effect depending on the part of the heat-generating portion.

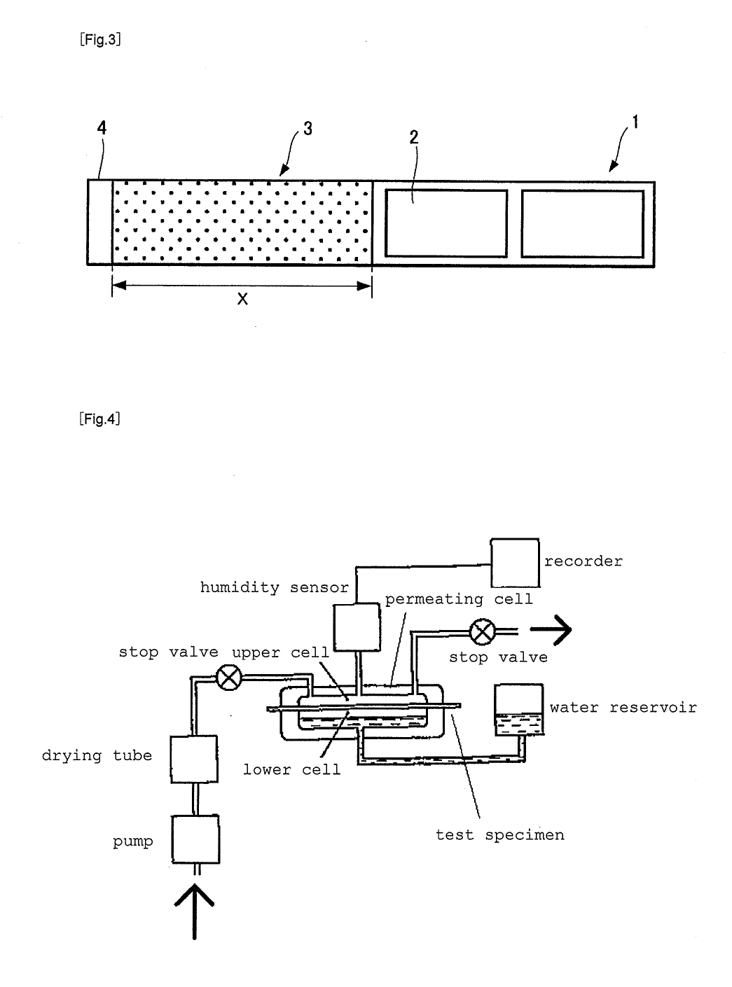

In the heating device of the invention, the band portion is connected to an end of the heat-generating portion, as shown in FIG. 3.

A specific structure of the heating device of the invention is described below.

Heat-Generating Portion

The heat-generating portion encloses the exothermic composition in one or more sections, and preferably in two sections. Particularly, the heat-generating portion enclosing the exothermic composition in two sections can be advantageously brought into close contact with the body, thereby effectively imparting a heating effect to the affected area. FIG. 3 shows a heating device including a heat-generating portion that encloses the exothermic composition in two sections.

The heat-generating portion may have any shape that can be advantageously brought into close contact with the body, such as, for example, a rectangular shape in which the sections can be arranged in the longitudinal direction.

The size of the heat-generating portion is not limited; for example, when the heat-generating portion is rectangular, it is preferable that the long side be about 10 to 40 cm, and the short side be about 5 to 20 cm; it is more preferable that the long side be about 20 to 30 cm, and the short side be about 6 to 15 cm.

The shape of each section is not limited, and may, for example, be rectangular, circular, or oval.

The size of each section may be suitably determined according to the size and the like of the heat-generating portion. For example, when the heat-generating portion has a rectangular shape in which rectangular sections are arranged in the longitudinal direction, the length of each section (along the short side of the heat-generating portion) is preferably about 4 to 15 cm, and more preferably about 5 to 12 cm; and the width of each section (along the long side of the heat-generating portion) is preferably about 2 to 8 cm, and more preferably about 3 to 6 cm.

The amount of the exothermic composition enclosed in each section may be within a range such that the heating effect can be sufficiently demonstrated, and is suitably determined according to the composition of the exothermic composition. Typically, the weight per unit area of the exothermic composition in each section is about 0.11 to 0.94 g/cm², and preferably about 0.15 to 0.71 g/cm². The weight per unit area of the exothermic composition in each section herein represents the value obtained by dividing the weight (g) of the exothermic composition enclosed in each section by the area (cm²) of the face of the section provided in the heat-generating portion that is brought into contact with the body.

When the heat-generating portion includes two or more sections, the distance between adjacent sections is not limited, and may be suitably determined according to the size and the like of the heat-generating portion. The distance is preferably about 0.5 to 4 cm, and more preferably about 0.5 to 2.5 cm. The distance between adjacent sections herein represents the shortest distance between a pair of the closest adjacent sections.

The heat-generating portion includes a first sheet (a layer that is brought into contact with the body) and a second sheet (a layer that is brought into contact with the band portion) laminated to the first sheet. The exothermic composition is enclosed in the sections provided between the first and second sheets.

(1) First Sheet

The first sheet is not limited, as long as it is a non-breathable film or sheet that is generally used as a packaging material of chemical warmers. A single or laminated film or sheet is used alone or in combination with a woven or nonwoven fabric.

Typically, a thermoplastic synthetic resin or the like is used as the resin that forms the film. Specific examples of preferable thermoplastic synthetic resins include polyethylenes, polypropylenes, polyesters, polyamides, polyvinyl alcohols, polyvinyl chloride, polyvinylidene chloride, polyurethanes, polystyrenes, ethylene-vinyl acetate copolymer, polycarbonates, and rubber hydrochloride. These resins are used alone or in combination. Polyethylenes are particularly preferable as the resin that forms the film.

When a laminated film or sheet is used as the first sheet, it is typically produced by lamination or other methods. Lamination may be performed according to any known method. For example, lamination may be performed by a method using thermal bonding, or an adhesive such as a hot melt adhesive or an acrylic or urethane adhesive. The surfaces of the laminated film or sheet may be completely bonded, or partially bonded to maintain flexibility.

Examples of nonwoven fabrics that may be laminated to the above-mentioned film include nonwoven fabrics containing synthetic fibers such as nylon, vinylon, polyesters, polyethylene terephthalate, rayon, acetate, acrylics, polyethylenes, polypropylenes, and polyvinyl chloride; and nonwoven fabrics containing natural fibers such as cotton, hemp, and silk. Polyethylene terephthalate is particularly preferable as the nonwoven fabric to be laminated. The nonwoven fabric has a weight of about 20 to 100 g/m².

(2) Second Sheet

The second sheet may be a sheet having a moisture permeability of 340 to 610 g/m² per day, as measured based on Method A (Humidity Sensor Method) defined in JIS K7129.

The second sheet may be a film or sheet that is breathable on all of the surfaces thereof. Typically, a single or laminated porous film or sheet is used alone or in combination with a woven or nonwoven fabric.

Typically, a thermoplastic synthetic resin is used as the resin that forms the film. Specific examples of preferable thermoplastic synthetic resins include polyethylenes, polypropylenes, polyesters, polyamides, polyvinyl alcohols, polyvinyl chloride, polyvinylidene chloride, polyurethanes, polystyrenes, ethylene-vinyl acetate copolymer, polycarbonates, and rubber hydrochloride. These resins are used alone or in combination. Polyethylenes are particularly preferable as the resin that forms the film.

An oriented film, which is preferably an oriented porous film, or a sheet including the film, is advantageously used as the breathable film. The oriented porous film, which typically contains an inorganic filler such as calcium carbonate, is made breathable upon formation of pores by drawing. The air permeability of the film can be controlled by controlling the pore size. The breathable film is preferably a laminated olefin-based (particularly a polyethylene-based) oriented and porous film, or a composite sheet thereof with a nonwoven fabric.

When a laminated film or sheet is used as the first sheet, it is typically produced by lamination or other methods. Lamination may be performed according to any known method. For example, lamination may be performed by a method using thermal bonding, or an adhesive such as a hot melt adhesive or an acrylic or urethane adhesive. The surfaces of the laminated film or sheet may be completely bonded, or partially bonded to maintain flexibility.

Examples of nonwoven fabrics that may be laminated to the film include nonwoven fabrics containing synthetic fibers such as nylon, vinylon, polyesters, rayon, acetate, acrylics, polyethylenes, polypropylenes, and polyvinyl chloride; and nonwoven fabrics containing natural fibers such as cotton, hemp, and silk. The nonwoven fabric has a weight of about 20 to 100 g/m².

(3) Exothermic Composition

The exothermic composition enclosed in bags may be any composition that generates heat by contact with air. Specifically, in the invention, a composition containing an iron powder, a water-retaining agent, a metal salt, and water is preferably used as the exothermic composition.

The total mass of the iron powder, water-retaining agent, metal salt, and water in the exothermic composition is preferably about 80 to 100 mass %.

The exothermic composition is described in detail below, taking an exothermic composition containing an iron powder, a water-retaining agent, a metal salt, and water as a representative example.

Iron Powder

The heating device of the invention can exhibit a heating effect when the iron powder generates heat by reacting with oxygen in the air.

Examples of the iron powder include reduced iron and cast iron. These iron powders can be used alone or in combination.

The iron powder may be in the form of granules, fibers, or the like. These forms of iron powders may be used alone or in combination.

The particle size of the granular iron powder is typically within the range of about 10 to 300 pin, and preferably about 10 to 100 μm.

The particle size as referred to herein can be determined as follows: One hundred grams of the test specimen (the iron powder or the like) to be measured is placed in an electric vibrating screen including, sequentially from the top, screens of 700 μm, 650 μm, 500 μm, 400 μm, 300 μm, 250 μm, 100 μm, 50 μm, and 10 μm, and vibrated for 15 minutes. The particle size can be subsequently determined by measuring the amount of the test specimen remaining in each screen and the amount of the test specimen passed through each screen. For example, when an iron powder with a particle size of 10 to 300 μm is used, an iron powder that has completely passed through the 300 μm screen, and remains on any of, or all of the 10 to 250 μm screens, may be used.

The amount of the iron powder in the exothermic composition is preferably about 30 to 80 mass %, and more preferably about 45 to 65 mass %.

Water-Retaining Agent

In the invention, the water-retaining agent is a material capable of holding water. Examples of water-retaining agents include porous materials and water-absorbing resins.

Specific examples of porous materials used as the water-retaining agent include activated carbon, wood flour, perlite, and vermiculite.

Activated carbon is capable of incorporating air into micropores on the surface to promote oxygen supply, or of retaining heat so that the heat-release temperature does not vary. Activated carbon has a very porous inside structure, and therefore provides a particularly good water-retaining ability. Furthermore, activated carbon absorbs well not only water, but also water vapor that evaporates upon the generation of heat from the exothermic composition, thereby preventing the water vapor from escaping. Thus, activated carbon can also be very useful as a water-retaining material. Activated carbon can also absorb odor emitted by the oxidation of the iron powder. Activated carbon prepared from, for example, coconut husks, wood, charcoal, coal, and bone black can be preferably used as the activated carbon. The activated carbon may be in the form of granules, fibers, or the like. These forms of activated carbon may be used alone or in combination. Specifically, in the invention, granular activated carbon is preferably used. When granular activated carbon is used, the particle size is preferably about 10 to 300 μm, and more preferably about 10 to 100 μm. The particle size of the activated carbon is measured in the same manner as the particle size of the iron powder.

Wood flour, perlite, and vermiculite may also take any form as long as they can retain water, but are preferably in granular form to improve the feeling of use of the heating device.

When wood flour, perlite, or vermiculite in granular form is used, the particle size is typically about 300 μm or less, and preferably about 250 μm or less. The particle size of wood flour, perlite, or vermiculite is also measured in the same manner as the particle size of the iron powder.

Among these porous materials, activated carbon, and vermiculite are preferably used; activated carbon and vermiculite are more preferably used; and activated carbon is particularly preferably used. These porous materials may be used alone or in combination.

Specific examples of water-absorbing resins used as the water-retaining agent include isobutylene-maleic anhydride copolymer, polyvinyl alcohol-acrylic acid copolymer, starch-acrylate graft copolymer, a polyacrylate crosslinked product, acrylate-acrylic ester copolymer, acrylate-acrylamide copolymer, and a crosslinked polyacrylonitrile. Preferable among these water-absorbing resins is a polyacrylate crosslinked product. The particle size of the water-absorbing resin is typically about 100 to 500 μm, and preferably about 250 to 400 μm. The particle size of the water-absorbing resin is also measured in the same manner as the particle size of the iron powder.

These water-absorbing resins can be used alone or in combination.

The porous materials and water-absorbing resins may be used alone or in combination as the water-retaining agent. The water-retaining agent used in the exothermic composition is preferably a porous material, or a combination of a porous material and a water-absorbing resin; more preferably activated carbon, or a combination of activated carbon, a porous material (other than activated carbon), and a water-absorbing resin; and still more preferably a combination of activated carbon, vermiculite, and a polyacrylate crosslinked product.

The amount of the water-retaining agent in the exothermic composition is preferably about 2 to about 30 mass %, and more preferably about 5 to about 20 mass %. More specifically, when a porous material is used alone as the water-retaining agent, the amount of the porous material in the exothermic composition is preferably 10 to 30 mass %, and more preferably about 10 to 20 mass %. When a water-absorbing resin is used alone as the water-retaining agent, the amount of the water-absorbing resin in the exothermic composition is preferably 2 to 10 mass %, and more preferably about 2 to 7 mass %. When a combination of a porous material and a water-absorbing resin is used as the water-retaining agent, the amounts of the porous material and water-absorbing resin in the exothermic composition are preferably 5 to 20 mass % and 1 to 10 mass %, respectively, and more preferably 7 to 20 mass % and 1 to 5 mass %, respectively. Particularly when a combination of activated carbon, a porous material other than activated carbon, and a water-absorbing resin is used as the water-retaining agent, the amounts of the activated carbon, porous material, and water-absorbing resin are preferably 3 to 20 mass %, 1 to 10 mass %, and 1 to 10 mass %, respectively, and more preferably 5 to 15 mass %, 1 to 5 mass %, and 1 to 5 mass %, respectively.

Metal Salt

A metal salt facilitates the oxidation reaction with air, and therefore activates the surface of the iron powder to promote the oxidation reaction of the iron.

A metal salt used in known exothermic compositions may be used as the metal salt. Examples of such metal salts include sulfates such as ferric sulfate, potassium sulfate, sodium sulfate, manganese sulfate, and magnesium sulfate; and chlorides such as cupric chloride, potassium chloride, sodium chloride, calcium chloride, manganese chloride, magnesium chloride, and cuprous chloride. Carbonates, acetates, nitrates, and other salts can also be used. These metal salts can be used alone or in combination.

The particle size of the metal salt is typically about 100 to 700 μm, and preferably about 250 to 650 μm. The particle size of the metal salt is also measured in the same manner as the particle size of the iron powder.

The amount of the metal salt in the exothermic composition is preferably about 0.5 to 10 mass %, and more preferably about 1 to 3 mass %.

Water

Usable types of water include distilled water and tap water. The amount of water in the exothermic composition is preferably about 1 to 40 mass %, and more preferably about 20 to 30 mass %.

Other Additives

In addition to the above-described components, the exothermic composition may optionally contain other additives that can be used in exothermic compositions.

Mixing the Components

The exothermic composition can be prepared by mixing the above-described components. Mixing may be performed under vacuum or an inert gas atmosphere, as required. Mixing may be performed according to, for example, the method described in U.S. Pat. No. 4,649,895.

(4) Preparation of Heat-Generating Portion

The heat-generating portion is obtained by bonding the first and second sheets so that the exothermic composition is enclosed in one or more sections. When a laminate is used as each of the first and second sheets, the first and second sheets are bonded to each other so that the nonwoven fabric that forms each laminate faces outside (i.e., opposite the face that comes into contact with the enclosed exothermic composition). At this moment, in order to form each section that encloses the exothermic composition, all of the regions of the heat-generating portion excluding each section are bonded. For example, in FIG. 3, the first and second sheets are bonded to each other in the regions of the heat-generating portion 1 except for the sections 2.

Examples of usable bonding methods include, but are not limited to, thermal bonding and methods of bonding using resin components as mentioned.

Elastic Band

The band portion can surround all or a portion of the heat-generating portion to keep the heat-generating portion in contact with the body. As shown in FIG. 3, in the heating device of the invention, the heat-generating portion 1 is connected to one end of the elastic band portion 3 along the direction of elasticity X. Although the band portion 3 is connected to only one end of the heat-generating portion 1, the band portion 3 may also be connected to both ends of the heat-generating portion 1. Two or more band portions may also be connected to one end of the heat-generating portion. Examples of connecting methods include, but are not limited to, bonding with a known adhesive, fixing with a thread, ultrasonic welding, and other methods. Moreover, as shown in FIG. 3, the other end of the band portion 3 along the direction of elasticity X is typically provided with a bonded portion 4 that provides excellent adhesion with the heat-generating portion and/or the band portion, in order to keep the heat-generating portion in contact with the body. The bonded portion 4 may, for example, be MAGICTAPE (registered trademark).

The size of the band portion can be suitably adjusted according to the size of the affected area to which the heating device is applied. The size of the band portion is not limited as long as it can advantageously keep the heat-generating portion in contact with the body. The size of the band portion is preferably such that the long side is about 10 to 40 cm, and the short side is about 5 to 20 cm; and more preferably, the long side is about 20 to 30 cm, and the short side is about 6 to 15 cm. When the band portion has a size such that the long side is about 10 to 40 cm, and the short side is about 5 to 20 cm, the heat-generating portion can be advantageously brought into close contact with the body.

The elongation of the band portion is not limited as long as it is within a range such that the band portion can surround the heat-generating portion to advantageously keep the heat-generating portion in contact with the body.

The specific structure of the band portion is not limited as long as the band portion has an air permeability of 3 s/300 cc to 61 s/300 cc, as measured according to JIS P 8117-1998: “Paper and board—Determination of air permeance and air resistance: Gurley method”, and has a certain degree of elasticity. When the band portion has an air permeability within this range, it can supply a sufficient amount of air to the heat-generating portion, allowing the heating device to provide desired heating and moisturizing effects. The air permeability of the band portion is preferably 3 s/300 cc to 61 s/300 cc, more preferably about 3 s/300 cc to 8 s/300 cc, still more preferably about 5 s/300 cc to 8 s/300 cc, and particularly preferably about 7 s/300 cc.

The material of the band portion is preferably a nonwoven or woven fabric of a natural or synthetic fiber, in order to impart elasticity to the band portion.

Embodiments of the Use of the Heating Device

The heat-generating portion (the exothermic composition) that forms the heating device of the invention generates heat in the presence of air. Therefore, in order to avoid contact with air, the heating device is typically distributed while being enclosed in an airtight package.

The heating device of the invention allows the heat-generating portion to come into contact with the body, thereby imparting a heating effect to the affected area. Because the heat-generating portion is fixed with the elastic band portion, the heating device of the invention is not easily shifted from the affected area (the worn position). Moreover, the heating device of the invention can typically maintain the temperature at about 38 to 42° C.

Thus, the heating device of the invention can ameliorate diseases and symptoms of the thumb side of a wrist joint, a wrist, an ankle, a knee, an elbow, a neck, and the like. Furthermore, the heating device of the invention can be advantageously used as a device for rehabilitation after treatment. The heating device can be advantageously fixed, for example; on the thumb side of a wrist joint, and is therefore effective for the treatment of de Quervain's disease.

Furthermore, in the heating device of the invention, the face of the heat-generating portion that is brought into contact with the band portion has a moisture permeability within the above-defined range, and the band portion has an air permeability within the above-defined range. This effectively imparts moisture along with a warm sensation to the area of the body in contact with the heating device. That is, the heating device can also exhibit moisturizing effects.

Because of its excellent heating effect, the heating device of the invention can be advantageously used not only for treatment and rehabilitation, but also as a body warmer.

Method for Manufacturing the Heating Device

The heating device can be manufactured by, for example, a method including the following steps:

forming a heat-generating portion by laminating a first sheet that is non-breathable and a second sheet that is breathable so that an exothermic composition that generates heat by contact with air is enclosed in one or more sections; and

connecting an elastic band portion to an end of the heat-generating portion formed in the previous step so that the elastic band portion encloses all or a portion of the heat-generating portion, allowing the heat-generating portion to be kept in contact with the body.

Accordingly, the invention also provides a method for easily manufacturing the above-described heating device that can stably provide an excellent heating effect over an extended period of time. In the method of the invention, the first sheet, second sheet, exothermic composition, and elastic band portion according to the above-described embodiments may be suitably employed.

### EXAMPLES

Referring to the following Examples and Comparative Examples, the invention is described in greater detail below; however, the invention is not limited to these Examples.

### Examples and Comparative Examples

A heating device having the structure shown in FIG. 3 was prepared.

Heat-Generating Portion

1) Exothermic Composition

An exothermic composition was prepared by mixing an iron powder with a particle size of 50 μm, activated carbon with a particle size of 200 μm, salt with a particle size of 380 μm, water, vermiculite with a particle size of 100 μm, and an acrylic acid polymer partial sodium salt crosslinked product with a particle size of 380 μm. The amounts of the iron powder, activated carbon, sodium chloride, water, vermiculite, and sodium polyacrylate in the exothermic composition were 55 mass %, 13 mass %, 1 mass %, 26 mass %, 3 mass %, and 2 mass %, respectively.

The exothermic composition was prepared according to the method described above.

2) First Sheet

First, a nonwoven fabric (weight: 30 g/m²) produced by spunlace using polyethylene terephthalate was laminated to a film containing polyethylene as a resin component, thereby preparing a laminate with a length of 8 cm and a width of 27.5 cm.

The first sheet was prepared according to the method described above.

3) Second Sheet

A fiber sheet (weight: 30 g/m²), which was made into a composite fiber by thermal bonding using polypropylene and polyethylene, was laminated to a porous film (thickness: 70 μm), which was made porous by drawing a film principally containing an olefin-based resin and an inorganic filler (calcium carbonate).

During the formation of the porous film, the moisture permeability of the second sheet was adjusted to be in the range of 282 to 634 g/m² per day by adjusting the amount of the calcium carbonate.

Eighteen types of second sheets were prepared according to the method described above.

The moisture permeability was measured according to Method A (Humidity Sensor Method) defined in JIS K7129. Method A (Humidity Sensor Method) defined in JIS K7129 was performed as follows: One side of a test specimen (the second sheet) was saturated with water vapor, and the opposite side was adjusted to a predetermined relative humidity. A change in humidity due to the amount of water vapor passed through the test specimen was detected with a humidity sensor installed above the low-humidity side of the test specimen, and the detected change was converted to an electrical signal. The water vapor passage time required until a certain range of relative humidities was reached was measured. After confirming that the water vapor passage rate had reached a steady state, the water vapor permeability was calculated based on the measured value. More specifically, the measurement according to Method A was performed as follows.

In the measurement according to Method A, a test specimen with a known water vapor permeability was used as a reference specimen. Additionally, using a pre-conditioned test specimen, test specimen conditioning was carried out prior to the test for 88 hours or more under the second grade of reference temperature and humidity conditions as defined in JIS K 7100 (temperature: 23±2° C.; relative humidity (50±5) %). The test specimen must be free of defects such as wrinkles, creases, and pinholes, as well as uniform in thickness. The test specimen was prepared by cutting a portion satisfying these requirements into a size of 15 to 10.5 cm.

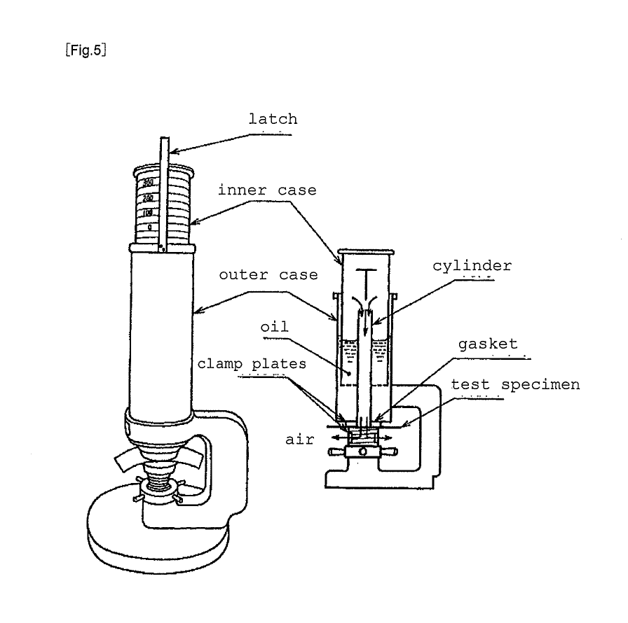

A water vapor permeability measuring apparatus was used for the measurement according to Method A. The apparatus mainly included a permeating cell having two measurement cells disposed on upper and lower sides of the test specimen, the upper cell having a low humidity and the lower cell having a high humidity; a humidity sensor for detecting the permeated water vapor as a relative humidity; a pump and a drying tube for feeding dry air; and a water reservoir. FIG. 4 shows one example of the water vapor permeability measuring apparatus. The testing conditions were as follows: a test temperature of 40±0.5° C., and a relative humidity (RH) of 90±2%.

The measurement was performed according to the following procedure: A certain amount of distilled water was sealed in the lower cell, and the reference specimen or test specimen was placed between the upper and lower cells in such a manner that the specimen did not become wrinkled or sagged. The humidity inside the upper cell was subsequently reduced at or below 10% RH using dry air, and the measurement was started. An increase in relative humidity due to the water vapor passed through the specimen was detected with the humidity sensor, and the measurement was repeated until the time required for the unit relative humidity range to be reached as the amount of water vapor increased, stabilized to a fixed value within ±5%.

Based on the measured value, the water vapor permeability was calculated according to the following equation:

$$WVTR = \frac{S \times C}{T} \times F \qquad [ Equation 1 ]$$

wherein:

WVTR represents the water vapor permeability [g/(m²·24 h)] of the test specimen;

S represents the water vapor permeability [g/(m²·24 h)] of the reference specimen;

C represents the time (s) required for the unit relative humidity range of the reference specimen to be reached;

T represents the time (s) required for the unit relative humidity range of the test specimen to be reached; and

F represents the permeation area of the reference specimen/the permeation area of the test specimen.

4) Preparation of Heat-Generating Portion

A rectangular heat-generating portion having a long side of 27.5 cm and a short side of 8 cm was prepared by using the exothermic composition, the first sheet, and each of the second sheets.

Specifically, the heat-generating portion was prepared by forming two rectangular sections that enclosed the exothermic composition, and bonding all of the regions of the heat-generating portion except for the sections on the first and second sheets, in such a manner that the polyethylene resin film of the first sheet and the polyethylene resin film of the second sheet came into contact with each other. Each section had a length (along the short side of the heat-generating portion) of 6.5 cm and a width (along the long side of the heat-generating portion) of 10 cm. Bonding of the first sheet and second sheet was performed at 130° C. by thermal bonding. The filling ratio of the exothermic composition in each section was 30%.

Multiple heat-generating portions were prepared according to the above-described method.

Elastic Band

An elastic band portion 8 cm long and 20 cm wide (tradename “Optiflex”; Golden Phoenix Fiberwebs Inc.) was provided with uniform perforations at regular intervals using a 0.5 mm-wide drill, thereby preparing an elastic band portion having an air permeability of 1 to 74 s/300 cc.

The air permeability was measured according to JIS P 8117-1998: “Paper and board—Determination of air permeance and air resistance: Gurley method” (tester: type B). More specifically, the measurement was performed according to the method described below.

A Gurley densometer (type B) consists of an outer case that is partially filled with oil; and an inner case that has an open or sealed top and is allowed to move vertically inside the outer case. An example of a specific structure of the densometer is given in FIG. 5. The air pressure required for the test depends on the mass of the inner case. The Gurley densometer (type B) is configured to apply air pressure to a test specimen that is held between a pair of clamp plates having inner holes with a diameter of 28.6±0.1 mm. The pair of clamp plates are attached to a base of the densometer. A gasket is disposed to come into contact with the clamp plate on the surface subjected to compressed air, and is configured to bring the clamp plate into contact with the test specimen and clamp them together.

The air permeability (Gurley) can be measured using the Gurley densometer as follows: Air is compressed by the weight of the vertical inner case floating on a fluid. The air permeability can be determined by measuring the time required for a certain amount of the compressed air to pass through the specimen. Specifically, the air resistance (Gurley) represents the time required for 300 cc of air to pass through paper or board with an area of 642 mm².

For measuring the air permeability using the Gurley densometer, a test specimen was prepared by selecting a portion free of defects such as wrinkles and creases, and cutting the portion into a size of 50×50 mm² or more.

The measurement was performed according to the following procedure: The densometer was horizontally placed so that the inner case stood vertically. The outer case was filled with oil up to a reference line of approximately 120 mm. First, the inner case was pulled up until its top was supported by a latch. Next, the test specimen was clamped between the clamp plates, and the inner case was slowly lowered until it floated. When the inner case was in the stationary state, the time required for the reference line from 0 to 300 cc to pass the edge of the outer case was measured. The test was conducted on at least the front and back of five test specimens of each of the heat-generating portions. The average value of the results was determined as the air permeability (s/300 cc).

The elastic band portion was provided with MAGICTAPE (registered trademark) 7 cm long and 2 cm wide.

Heating Device

A heating device was prepared by ultrasonically fixing the elastic band portion to an end of each heat-generating portion. In the Examples and Comparative Examples, heating devices were prepared by suitably combining the elastic band portion with twelve types of heat-generating portions.

Each of the heating devices prepared in the Examples and Comparative Examples was sealed in a bag made of a polyvinylidene chloride-coated film (KOP), in order to avoid contact with air.

Test Examples 1 to 3 given below were conducted immediately after the removal of the heating devices from the bag of the polyvinylidene chloride-coated film.

### Test Example 1

### (1) Examination of Contact Area between Heat-Generating Portion and Band, as well as Moisture Permeability of Second Sheet of Heat-Generating Portion

Heating devices were prepared using heat-generating portions made using the second sheets having moisture permeabilities of 282 to 634 g/m² per day and a band portion having a moisture permeability of 7 s/300 cc. These heating devices were applied to healthy subjects to evaluate their heating effect. Specifically, as shown in FIG. 1, in order to keep the first sheet-side of the heat-generating portion in contact with the wrist, each heat-generating portion was surrounded by the band portion so that 10%, 30%, 60%, or 100% of the region of the heat-generating portion was covered with the band portion, and the heat-generating portion was fixed with the MAGICTAPE (registered trademark) of the band portion.

The heating effect was evaluated as follows. The skin temperature of the portion of the wrist in contact with the heat-generating portion was measured at two points, using a thermo-recorder (tradename “RT-12”; Espec Test Center Corp.), and the average value of the measured temperatures was determined. The evaluation was made according to the following criteria:

Criteria

A: A skin temperature of 39 to 41° C.

B: A skin temperature of 38° C. to less than 39° C., or more than 41° C. to 42° C.

C: A skin temperature of less than 38° C. or more than 42° C.

The results are shown in Table 1.

This test was conducted on 10 healthy subjects; the evaluation results were the same for all 10 subjects.

TABLE 1
Moisture Permeability (g/m² per day)

| | | | | | |
|---|---|---|---|---|---|
| 634 | C | C | C | C | C |
| 605 | C | B | B | B | B |
| 550 | C | B | B | B | B |
| 527 | C | B | B | B | B |
| 502 | C | B | B | B | B |
| 468 | C | A | A | A | A |
| 451 | C | A | A | A | A |
| 424 | C | A | A | A | A |
| 398 | C | A | A | A | A |
| 370 | C | A | A | A | A |
| 363 | C | B | B | B | B |
| 347 | C | B | B | B | B |
| 338 | A | C | C | C | C |
| 331 | A | C | C | C | C |
| 322 | A | C | C | C | C |
| 309 | A | C | C | C | C |
| 300 | A | C | C | C | C |
| 282 | A | C | C | C | C |
| | 0 | 10 | 30 | 60 | 100 |

Contact Region (%) of the Band on the Heat-Generating Portion

The results shown in Table 1 revealed that a heating effect providing a skin temperature of 38° C. to less than 39° C., or more than 41° C. to 42° C. can be achieved when the proportion of the contact region of the elastic band portion on the heat-generating portion of the heating device is 10 to 100%, and the second sheet has a moisture permeability of 347 to 605 g/m² per day.

The results also revealed that a superior heating effect providing a skin temperature of 39 to 41° C. can be achieved when the proportion of the contact region of the elastic band portion on the heat-generating portion of the heating device is 10 to 100%, and the second sheet has a moisture permeability of 370 to 468 g/m² per day.

### (2) Examination of Moisture Permeability of Second Sheet of Heat-Generating Portion and Air Permeability of Band

Heating devices were prepared using heat-generating portions made using the second sheets having moisture permeabilities of 282 to 634 g/m² per day and a band portion having a moisture permeability of 1 to 74 s/300 cc. These heating devices were applied to healthy subjects to evaluate their heating effect. Specifically, each heat-generating portion was surrounded by the band portion so that 60% of the region of the heat-generating portion was covered with the band portion, and the heat-generating portion was fixed with the MAGICTAPE (registered trademark) of the band portion. Evaluations of the heating effect were subsequently conducted according to the same method as described above.

The results are shown in Table 2.

This test was conducted on 10 healthy subjects; the evaluation results were the same for all 10 subjects.

TABLE 2

| | | | | | | | | | | | | | | | |
|---|---|---|---|---|---|---|---|---|---|---|---|---|---|---|---|
| Air Permeability (s/300 cc) | 74 | C | C | C | C | C | C | C | C | C | C | C | C | C | C |
| | 61 | C | C | C | B | B | B | B | B | B | B | B | B | B | C |
| | 47 | C | C | C | B | B | B | B | B | B | B | B | B | B | C |
| | 32 | C | C | C | B | B | B | B | B | B | B | B | B | B | C |
| | 17 | C | C | C | B | B | B | B | B | B | B | B | B | B | C |
| | 10 | C | C | C | B | B | B | B | B | B | B | B | B | B | C |
| | 8 | C | C | C | B | B | A | A | A | A | B | B | B | B | C |
| | 7 | C | C | C | B | B | A | A | A | A | B | B | B | B | C |
| | 5 | C | C | C | B | B | A | A | A | A | B | B | B | B | C |
| | 3 | C | C | C | B | B | B | B | B | B | B | B | B | B | C |
| | 2 | C | C | C | C | C | C | C | C | C | C | C | C | C | C |
| | 1 | C | C | C | C | C | C | C | C | C | C | C | C | C | C |
| | | 282 | 331 | 338 | 347 | 363 | 370 | 424 | 451 | 468 | 502 | 527 | 550 | 605 | 634 |

Moisture Permeability (g/m² per day)

The results shown in Table 2 revealed that a good heating effect can be achieved when the moisture permeability of the second sheet of the heat-generating portion is 347 to 605 g/m² per day, and the air permeability of the band portion is 3 to 61 s/300 cc. The results also revealed that a remarkably good heating effect can be achieved particularly when the moisture permeability of the second sheet of the heat-generating portion is 370 to 468 g/m² per day, and the air permeability of the band portion is 5 to 8 s/300 cc.

Furthermore, even when the contact region between the heat-generating portion and the band portion was varied to 10%, 30%, and 100%, a good heating effect was obtained for all of the cases where the moisture permeability and air permeability were within the above-mentioned ranges; therefore, the same results as above were obtained.

### Test Example 2

Heating devices (Examples 1 to 6 and Comparative Examples 1 to 3) were prepared according to the same method as in Test Example 1, using heat-generating portions made using second sheets having moisture permeabilities of 370 to 634 g/m² per day and a band portion having a moisture permeability of 7 s/300 cc. These heating devices were applied to five patients suffering from joint pain caused by tenosynovitis. The heating effects of these heating devices were evaluated. Additionally, whether the pain was alleviated or not after wearing each heating device for 8 hours was evaluated according to the criteria given below. The test was conducted by surrounding the heat-generating portion by the band portion so that 60% of the region of the heat-generating portion was covered with the band portion.

Criteria for Determining Alleviation of Pain

A: Five patients felt that the pain was relieved.

B: Three or four patients felt that the pain was relieved.

C: One or two patients felt that the pain was relieved.

Nine heating devices listed in the following Table 3 were evaluated in Test Example 2. In this test example, the evaluation results “A” and “B” show that the effect of alleviating pain was obtained.

The results are shown in Table 3.

TABLE 3

| | | | | | | | Comp. | Comp. | Comp. |
|---|---|---|---|---|---|---|---|---|---|
| | Ex. 1 | Ex. 2 | Ex. 3 | Ex. 4 | Ex. 5 | Ex. 6 | Ex. 1 | Ex. 2 | Ex. 3 |
| Moisture | 370 | 424 | 468 | 347 | 502 | 605 | 282 | 338 | 634 |
| Permeability | | | | | | | | | |
| (g/m² per day) | | | | | | | | | |
| Heating Effect | A | A | A | B | B | B | C | C | C |
| Pain Alleviation | A | A | A | B | B | B | C | C | C |
| Effect | | | | | | | | | |

As shown in Table 3, the heating devices of the Examples were all found to provide superior heating effects, as well as the effect of alleviating joint pain. In particular, the heating devices of Examples 1 to 3, in which the moisture permeability of the second sheet was in the range of 370 to 468 g/m² per day, were found to provide even more superior heating effects and an effect of alleviating joint pain at the same time.

### Test Example 3

Ten subjects with dry hands wore, for 5 minutes, each of the heating devices of Examples 1 to 6 and Comparative Examples 1 to 3 shown in Test Example 2 so that 60% of the region of the heat-generating portion was covered with the band portion. Whether the heating devices provided moisturizing effects or not was evaluated.

The heating devices were worn as in Test Example 1. The results of the tests showed that all of the 10 subjects evaluated the heating devices of Examples 1 to 6 as having moisturizing effects after wearing them. That is, the heating devices of the Examples were found to have moisturizing effects as well as heating effects. On the other hand, after wearing the heating devices of Comparative Examples 1 to 3, the sensations of warmth and moisture were different between the portion of the heat-generating portion covered with the band portion and the portion of the heat-generating portion that was not covered with the band portion. Therefore, the perceived effects were nonuniform depending on the part of the heat-generating portion.

### REFERENCE SIGNS LIST

1. Heat-Generating Portion
2. Section
3. Elastic Band Portion
4. Bonded Portion
X. Direction of Elasticity

1. A heating device comprising:
a heat-generating portion having an exothermic composition enclosed in one or more sections, the exothermic composition generating heat by contact with air; and
an elastic band portion;
(1) the band portion surrounding all or a portion of the heat-generating portion to keep the heat-generating portion in contact with the body;
(2) the band portion being connected to an end of the heat-generating portion;
(3) a face of the heat-generating portion that is brought into contact with the band portion having a moisture permeability of 340 to 610 g/m² per day, as measured according to Method A (Humidity Sensor Method) defined in JIS K7129; and
(4) the band portion having an air permeability of 3 s/300 cc to 61 s/300 cc, as measured according to JIS P 8117-1998: “Paper and board—Determination of air permeance and air resistance: Gurley method”.

2. The heating device according to claim 1, wherein the band portion is made of a nonwoven fabric.

3. The heating device according to claim 1, wherein the exothermic composition contains an iron powder, a water-retaining agent, a metal salt, and water.

4. The heating device according to claim 1, wherein the exothermic composition contains 30 to 80 mass % of the iron powder, 2 to 30 mass % of the water-retaining agent, 0.5 to 10 mass % of the metal salt, and 1 to 40 mass % of the water; and the total amount of these components in the exothermic composition is 80 to 100 mass %.

5. The heating device according to claim 1, wherein the face of the heat-generating portion that is brought into contact with the band portion has a moisture permeability of 365 to 475 g/m² per day.

6. The heating device according to claim 1, wherein the band portion has an air permeability of 3 to 8 s/300 cc.

7. The heating device according to claim 1, which is a therapeutic or rehabilitation device for a thumb side of a wrist joint, a wrist, an ankle, a knee, an elbow, or a neck.

8. A method for manufacturing a heating device comprising:
forming a heat-generating portion by laminating a first sheet that is non-breathable and a second sheet that is breathable so that an exothermic composition that generates heat by contact with air is enclosed in one or more sections; and
connecting an elastic band portion to an end of the heat-generating portion formed in the previous step so that the elastic band portion encloses all or a portion of the heat-generating portion, allowing the heat-generating portion to be kept in contact with the body;
a face of the heat-generating portion that is brought into contact with the band portion having a moisture permeability of 340 to 610 g/m² per day, as measured according to Method A (Humidity Sensor Method) defined in JIS K7129; and
the band portion having an air permeability of 3 s/300 cc to 61 s/300 cc, as measured according to JIS P 8117-1998: “Paper and board—Determination of air permeance and air resistance: Gurley method”.

9. The heating device according to claim 2, wherein the exothermic composition contains an iron powder, a water-retaining agent, a metal salt, and water.

10. The heating device according to claim 2, wherein the exothermic composition contains 30 to 80 mass % of the iron powder, 2 to 30 mass % of the water-retaining agent, 0.5 to 10 mass % of the metal salt, and 1 to 40 mass % of the water; and the total amount of these components in the exothermic composition is 80 to 100 mass %.

11. The heating device according to claim 3, wherein the exothermic composition contains 30 to 80 mass % of the iron powder, 2 to 30 mass % of the water-retaining agent, 0.5 to 10 mass % of the metal salt, and 1 to 40 mass % of the water; and the total amount of these components in the exothermic composition is 80 to 100 mass %.

12. The heating device according to claim 2, wherein the face of the heat-generating portion that is brought into contact with the band portion has a moisture permeability of 365 to 475 g/m² per day.

13. The heating device according to claim 3, wherein the face of the heat-generating portion that is brought into contact with the band portion has a moisture permeability of 365 to 475 g/m² per day.

14. The heating device according to claim 4, wherein the face of the heat-generating portion that is brought into contact with the band portion has a moisture permeability of 365 to 475 g/m² per day.

15. The heating device according to claim 2, wherein the band portion has an air permeability of 3 to 8 s/300 cc.

16. The heating device according to claim 3, wherein the band portion has an air permeability of 3 to 8 s/300 cc.

17. The heating device according to claim 4, wherein the band portion has an air permeability of 3 to 8 s/300 cc.

18. The heating device according to claim 2, which is a therapeutic or rehabilitation device for a thumb side of a wrist joint, a wrist, an ankle, a knee, an elbow, or a neck.

19. The heating device according to claim 3, which is a therapeutic or rehabilitation device for a thumb side of a wrist joint, a wrist, an ankle, a knee, an elbow, or a neck.

20. The heating device according to claim 4, which is a therapeutic or rehabilitation device for a thumb side of a wrist joint, a wrist, an ankle, a knee, an elbow, or a neck.
